(19) United States
(12) Patent Application Publication
(10) Pub. No.: US 2015/0079146 A1
(43) Pub. Date: Mar. 19, 2015

(54) BIOACTIVE GLASS FIBER MESH FOR REPAIR OF HARD TISSUES

(71) Applicant: NOVABONE PRODUCTS, LLC., Alachua, FL (US)

(72) Inventors: Gregory J. Pomrink, Newberry, FL (US); Zehra Tosun, Gainesville, FL (US)

(73) Assignee: NOVABONE PRODUCTS, LLC., Alachua, FL (US)

(21) Appl. No.: 14/390,969

(22) PCT Filed: Apr. 5, 2013
(86) PCT No.: PCT/US13/35426

§ 371 Date: Oct. 6, 2014

Related U.S. Application Data
(60) Provisional application No. 61/623,357, filed on Apr. 12, 2012.
(60) Provisional application No. 61/641,961, filed on May 3, 2012.

(51) Int. Cl. A61L 27/12; A61F 2/28; A61F 2/00

(52) U.S. Cl. CPC A61L 27/12; A61F 2/0063; A61F 2/28; A61F 2002/2835
USPC 424/423; 424/602

(57) ABSTRACT

The invention relates to the use of a bioactive glass material. For example, an autograft chip is wrapped in bioactive glass material for the treatment of bone injuries and bone defects in the body. The material can be in one or more of several forms, such as fibers, a fiber mesh, a sheet, or another shape. The material can be applied to hard tissues, such as bone.

### CROSS-REFERENCE TO RELATED APPLICATIONS

This application claims the benefit of U.S. Provisional Patent Application No. 61/623,357, filed Apr. 12, 2012, and U.S. Provisional Patent Application No. 61/641,961, filed May 3, 2012, the entire contents of which are hereby incorporated herein by reference.

### BACKGROUND

This invention relates generally to autograft chips wrapped in bioactive glass compositions alone, as a graft extender, or to maintain the position of bone fragments, for at least the repair of bone injuries and bone defects.

There are many materials used today for the repair and regeneration of bone defects. Bone is a composite of collagen, cells, a form of calcium hydroxyapatite crystals, and small quantities of other proteins of organic molecules that has unique properties of high strength, rigidity, and ability to adapt to varying loads. When bone injuries occur, it is necessary to fill voids or gaps in the bone as well as to encourage the repair and regeneration of bone tissue. One such material used to fill voids and to encourage repair and regeneration is bioactive glass.

Bioactive glass was originally developed in 1969 by L. Hench. Additionally bioactive glasses were developed as bone replacement materials, with studies showing that bioactive glass can induce or aid in osteogenesis. Hench et al, J. Biomed. Mater. Res. 5:117-141 (1971). Bioactive glass can form strong and stable bonds with bone. Piotrowski et al., J. Biomed. Mater. Res. 9:47-61(1975). Further, bioactive glass is not considered toxic to bone or soft tissue from studies of in vitro and in vivo models. Wilson et al., J. Biomed. Mater. Res. 805-817 (1981).

The bonding of the glass to bone begins with the exposure of the glass to aqueous solutions. Sodium ions in the glass can exchange with hydronium ions in body fluids, which increases the pH. Calcium and phosphorous ions can migrate from the glass to form a calcium and phosphate-rich surface layer. Underlying this surface layer is another layer which becomes increasingly silica-, borate-, and/or strontium-rich due to the loss of sodium, calcium, and/or phosphate ions (see, e.g., U.S. Pat. No. 4,851,046). Hydrolysis may then disrupt the Si—O—Si bridges, B—O—B bridges, and/or Sr—O—Sr bridges in the layer to form silanol groups, boranol groups, and/or strontium hydroxide groups, which can disrupt the glass network. The glass network is then thought to form a gel in which calcium phosphate from the surface layer accumulates. Mineralization may then occur as calcium phosphate becomes crystalline hydroxyapatite, which effectively mimics the mineral layer of bones.

Bone grafting is another common way in which bone repair and regeneration may be enhanced. Autograft bone, usually taken from the iliac crest, is preferred over allograft bone because there is less risk of immune reaction, disease transmission, and graft collapse.

Application of bioactive glass to bone may promote bone remodeling. Bone remodeling occurs by an equilibrium between osteoblast-mediated bone formation and osteoclast-mediated bone destruction. When bone is injured or missing, such as in a fracture, promotion of osteoblast activity is thought to be helpful to induce bone formation. Further, promoting bone formation by osteoblasts may be helpful in locations in which there is significant bone loss in the absence of an apparent injury.

Glass fibers may be useful for wound healing. See, U.S. Patent Publication No. 2011/0165221 to Jung et al. Jung et al. also disclose a layer suitable for wrapping around an implant, such as a hip implant or bone repair implant.

Wraps and scaffolding are known to be useful for attachment to tissue and bone in need of repair. See, U.S. Patent Publication No. 2003/0193104 to Melican et al. and U.S. Patent Publication No. 2004/0267362 to Hwang et al.

Fabric bags are known for use in inserting material into discs of the spinal cord. See, U.S. Pat. No. 5,549,679.

### BRIEF SUMMARY

In one aspect, the invention provides for a composition and a method for wrapping an autograft chip applied to a bone defect. The autograft chip is wrapped with a bioactive glass ceramic. The ceramic may be in one or more of several forms, such as fibers, random fibers, oriented fibers, a fiber mesh, a sheet, or another shape such as glass wool, a ball of glass wool, a fabric, random-pressed cotton-like structures, wool-like structures, and/or randomly-oriented fibers. The bioactive glass ceramic provides structure, prevents the autograft chip from migrating from the bone defect, and provides for unexpectedly superior bone remodeling, osteoblast formation, or hydroxyapatite formation.

The bone defect may be a fracture or may result from an injury or bone disease, such as osteoporosis. The autograft chip wrapped in a bioactive glass ceramic may release one or more of silicate, calcium, and borate ions into the bone defect. Ions released into the bone defect can stimulate osteoblast activity. The bioactive glass fiber may further comprise a carrier, such as hydroxyapatite and tricalcium phosphate, or a graft extender.

Another aspect of the invention provides for a bone defect treatment wrap. The wrap is comprised of an autograft chip and a bioactive glass sheet. The bioactive glass sheet comprises a mesh of bioactive glass fibers. The bioactive glass sheet is wrapped around the autograft chip and the bone defect to prevent the autograft chip from migrating from the bone defect.

The bone defect may be a fracture or may result from an injury or bone disease, such as osteoporosis. The bioactive glass sheet may release one or more of silicate, calcium, phosphate, strontium, and borate ions into the bone defect. Ions released into the bone defect can stimulate osteoblast activity. The bioactive glass sheet may further comprise a carrier, such as hydroxyapatite and tricalcium phosphate, or a graft extender.

Another aspect of the invention provides for a method for treating a bone defect using an autograft chip secured to the bone near the site of the bone defect by wrapping the bone and the autograft chip with a bioactive glass sheet.

Another aspect of the invention provides for treating a bone defect using an expandable empty bag comprising a mesh of bioactive glass fibers. The area of bone having a defect is exposed. At least one cavity is formed in the bone at or near the bone defect, in which a portion of the at least one cavity defines an opening. An expandable empty bag comprising a mesh of bioactive glass fibers is inserted into the opening. The bag is packed with an autograft chip until the bag expands to form a self-retaining rigid shape. The bag may further be filled with cement. The bag packed with the autograft chip, mesh of bioactive glass fibers, and optionally cement, is effective to prevent these components from migrating into and/or backing up into the spinal cord.

Another aspect of the invention provides for a method for treating a comminuted or complex bone fracture. A bone at the site of the bone fracture is wrapped with a bioactive glass ceramic. The bioactive glass ceramic may be in the form of fibers, a fiber mesh, random fibers, oriented fibers, and a sheet. The ceramic may be wrapped completely around the bone such that the ceramic is secured to the bone and/or maintains the bone shape so as to prevent further fracturing. The bioactive glass ceramic may be in the form of a sheet. The bioactive glass ceramic may also be secured to the bone by a plate or a screw.

Other systems, methods, features and advantages of the invention will be, or will become, apparent to one with skill in the art upon examination of the following figures and detailed description. It is intended that all such additional systems, methods, features and advantages be within the scope of the invention, and be encompassed by the following claims.

### DETAILED DESCRIPTION OF THE PREFERRED EMBODIMENTS

Unless defined otherwise, all technical and scientific terms used herein have the same meaning as commonly understood by one of ordinary skill in the art to which this invention pertains.

One aspect of the invention provides for a method for wrapping an autograft chip applied to a bone defect. The autograft chip is wrapped with a bioactive glass ceramic in the shape of fibers, mesh, sheet, or other shape. The bioactive glass ceramic provides structure and prevents the autograft chip from migrating from the bone defect.

Autograft chips may be formed by harvesting bone from a secondary operate site within the patient. The bone may be harvested from the iliac crest of the pelvis, for instance. In particular, when a patient has osteoporosis, there is a need to limit the amount of bone harvested from the iliac crest or another portion of the patient's body. Thus, the invention provides for harvesting a small amount of bone in conjunction with the use of bioactive glass mesh, fibers, etc.

The autograft chip may be wrapped in the bioactive glass fiber, mesh, etc. The autograft chip may be cultivated in vitro while wrapped in the bioactive glass before transplantation into the patient. The autograft chip wrapped in bioactive glass is transplanted into the patient.

A bioactive glass ceramic material suitable for the present compositions and methods may have sodium, calcium, strontium, phosphorous, silica, and/or boron present, as well as combinations thereof. In some embodiments, sodium, boron, silica, strontium, and calcium may each be present in the compositions in an amount of about 1% to about 99%, based on the weight of the bioactive glass ceramic. In further embodiments, sodium, boron, strontium and calcium may each be present in the composition in about 1%, about 2%, about 3%, about 4%, about 5%, about 6%, about 7%, about 8%, about 9%, or about 10%. In certain embodiments, sodium, boron, and calcium may each be present in the composition in about 5 to about 10%, about 10 to about 15%, about 15 to about 20%, about 20 to about 25%, about 25 to about 30%, about 30 to about 35%, about 35 to about 40%, about 40 to about 45%, about 45 to about 50%, about 50 to about 55%, about 55 to about 60%, about 60 to about 65%, about 65 to about 70%, about 70 to about 75%, about 75 to about 80%, about 80 to about 85%, about 85 to about 90%, about 90 to about 95%, or about 95 to about 99%. Some embodiments may contain substantially one or two of sodium, calcium, strontium, phosphorous, silicate, and boron with only traces of the other(s). The term “about” as it relates to the amount of calcium phosphate present in the composition means +/−0.5%. Thus, about 5% means 5+/−0.5%.

The bioactive glass materials may further comprise one or more of a silicate, borosilicate, borate, phosphate, strontium, or calcium, including SrO, CaO, P₂O₅, PO₃, PO₄, SiO₂, and B₂O₃. An exemplary bioactive glass is 45S5, which includes 46.1 mol % SiO₂, 26.9 mol % Ca), 24.4 mol % Na₂O and 2.5 mol % P₂O₅. An exemplary borate bioactive glass is 45S5B1, in which the SiO₂ of 45S5 bioactive glass is replaced by B₂O₃.

Other exemplary bioactive glasses include 58S, which includes 60 mol % SiO₂, 36 mol % CaO and 4 mol % P₂O₅, and S70C30, which includes 70 mol % SiO₂ and 30 mol % CaO. In any of these or other bioactive glass materials of the invention, SrO may be substituted for CaO.

Bioactive glass that may also be suitable include glasses having about 40 to about 60 wt % SiO₂, about 10 to about 34 wt % Na₂O, up to about 20 wt % K₂O, up to about 5 wt % MgO, about 10 to about 35 wt % CaO, 0 to about 35 wt % SrO, up to about 20 wt % B₂O₃, and/or about 0.5 to about 12 wt % P₂O₅. The bioactive glass may additionally contain up to 10 wt % CaF₂. In a certain embodiment, the bioactive glass has the following composition 53 wt % SiO₂, 6 wt % Na₂O, 12 wt % K₂O, 5 wt % MgO, 20 wt % CaO, and 4 wt % P₂O₅.

The following composition, having a weight % of each element in oxide form in the range indicated, will provide one of several bioactive glass compositions that may be used to form a bioactive glass ceramic:

| | |
|---|---|
| SiO₂ | 0-86 |
| CaO | 4-35 |
| Na₂O | 0-35 |
| P₂O₅ | 2-15 |
| CaF₂ | 0-25 |
| B₂O₃ | 0-75 |
| K₂O | 0-8 |
| MgO | 0-5 |
| CaF | 0-35 |

The bioactive glass ceramic can be in the form of a three-dimensional compressible body of loose glass-based fibers in which the fibers comprise one or more glass-formers selected from the group consisting of P₂O₅, PO₃, PO₄, SiO₂, and B₂O₃. Some of the fibers have a diameter between about 100 nm and about 10,000 nm, and a length:width aspect ratio of at least about 10. The pH of the bioactive glass can be adjusted as-needed.

In some embodiments, the body comprises fibers having a diameter between about 100 nm and about 10,000 nm. The especially small diameter of these fibers renders them highly flexible so they form into the compressible body without breaking. In some embodiments the body includes fibers meeting these dimensional requirements in addition to other glass morphologies, such as fibers of other dimensions, microspheres, particles, ribbons, flakes or the like. The fibers may have a variety of cross section shapes, such as flat, circular, oval, or non-circular.

When in the form of fibers, fiber mesh, random fibers, oriented fibers, or a sheet, the bioactive glass is also suitable for wrapping around an autograft chip that may then be applied to a bone injury or bone defect. This assists in fixation of the autograft chip in the body. In these applications where the glass body is a sheet, it is flexible and may have randomly oriented as well as woven fibers.

The bioactive glass may have osteoproductive properties, which refers to promoting proliferation of the osteoblasts such that bone can regenerate. In an osteoproductive process, a bioactive glass material may be colonized by osteogenic stem cells. This may lead to quicker filling of bone defects than would otherwise occur with an osteoconductive glass.

Bioactive glass ceramics may be prepared by heating a composition comprising one or more of SiO₂, CaH(PO₄), CaO, P₂O, Na₂O, CaCO₃, Na₂CO₃, K₂OO₃, MgO, and H₂BO₃ to a temperature between 1300 and 1500° C. such that the composition may form molten glass. An exemplary composition that can form fibers includes 40-60% SiO₂, 10-20% CaO, 0-4% P₂O₅, and 19-30% NaO. Other exemplary compositions include 45S5, which includes 46.1 mol % SiO₂, 26.9 mol % Ca), 24.4 mol % Na₂O and 2.5 mol % P₂O₅; 45S5B1, which includes 46.1 mol % B₂O₃, 26.9 mol % Ca), 24.4 mol % Na₂O and 2.5 mol % P₂O₅; 58S, which includes 60 mol % SiO₂, 36 mol % CaO and 4 mol % P₂O₅; and S70C30, which includes 70 mol % SiO₂ and 30 mol % CaO. Another exemplary composition includes 40 mol % SiO₂, 40 mol % B₂O₃, 20 mol % CaO, and 20 mol % Na₂O.

Bioactive glass ceramics, mesh, and/or fibers may be prepared by a sol-gel method. Methods of preparing such bioactive active glasses are described in Pereira, M. et al., “Bioactive glass and hybrid scaffolds prepared by sol-gel method for bone tissue engineering” Advances in Applied Ceramics, 2005, 104(1): 35-42 and in Chen, Q. et al., “A new sol-gel process for producing Na₂O-containing bioactive glass ceramics” Acta Biomaterialia, 2010, 6(10):4143-4153.

The composition can be allowed to solidify. In some embodiments, particles of bioactive glass are sintered to form a porous glass.

As the term “substantial amount of polymer” is used herein, it is intended to mean any amount that would provide structural integrity to the resultant bioactive glass structure.

Repeated cooling and reheating may be performed on the solidified or sintered bioactive glass, with or without spinning, to draw the bioactive glass produced into fibers. A glass drawing apparatus may be coupled to the spinner and the source of molten bioactive glass, such as molten bioactive glass present in a crucible, for the formation of bioactive glass fibers. The individual fibers can then be joined to one another, such as by use of an adhesive, to form a mesh. Alternatively, the bioactive glass in molten form may be placed in a cast or mold to form a sheet or another desired shape.

The bioactive glass fiber mesh or sheets may further comprise any one or more of adhesives, grafted bone tissue, in vitro-generated bone tissue, collagen, calcium phosphate, stabilizers, disinfectants, pigments, X-ray contrast media, fillers, and other materials that facilitate grafting of bioactive glass to bone.

The wrapped autograft chip may be administered surgically. For example, the chip may be inserted by implantation. In particular, the bioactive glass may be introduced into or onto a bone fracture or a damaged region of the bone. The bioactive fiber mesh random fibers, oriented fibers, and/or sheets may also be introduced into a bone defect or onto a bone defect.

In some embodiments, the bioactive glass sheet, fiber, or mesh is treated with certain buffer solutions to prepare the surface of the glass, fibers, or mesh for cell adhesion and controls pH prior to the exposure of the particles with cells. In this context, the bioactivity and bone formation using the glass, fibers, mesh, or ceramic of the present invention may be enhanced by treating these with a buffer solution.

In certain embodiments, the pre-treatment buffer solution has a starting pH of from about 6 to about 12 but may reach an end pH of about 9.5. Examples of buffers that might be suitable for the pre-treatment of the present invention include mixed sodium phosphate salts (such as Sorensen's Phosphate buffer, Millonig's Phosphate buffer, Karlsson and Shultz Phosphate buffer, Maunsbach Phosphate buffer, and Phosphate Buffered Saline (PBS); buffer pH of about 6.4-8.0), TAPS (3-{[tris(hydroxymethyl)methyl]amino}propanesulfonic acid; buffer pH of about 7.7-9.1), Bicine (N,N-bis(2-hydroxyethyl)glycine; buffer pH of 7.6-9.0), Tricine (N-tris(hydroxymethyl)methylglycine; buffer pH about 7.4-8.8), Tris(tris(hydroxymethyl)methylamine; buffer pH of about 7.5-9.0), HEPES (4-2-hydroxyethyl-1-piperazineethanesulfonic acid; buffer pH of about 6.8-8.2), TES (2-{[tris(hydroxymethyl)methyl]amino}ethanesulfonic acid; buffer pH of about 6.8-8.2), MOPS (3-(N-morpholino)propanesulfonic acid; buffer pH of about 6.5-7.9), PIPES (piperazine-N,N′-bis(2-ethanesulfonic acid); buffer pH of about 6.1-7.5), Cacodylate (dimethylarsinic acid; buffer pH of about 5.0-7.5), SSC (saline sodium citrate; buffer pH of about 6.5-7.5), or MES (2-(N-morpholino)ethanesulfonic acid; buffer pH of about 5.5-6.7). Any other buffer having appropriate pH buffering range of about 6 to about 12 might be suitable.

In certain embodiments, the end pH does not exceed 9.5, 9.4, 9.3, 9.2, 9.1, 9.0, 8.8, 8.9, 8.7, 8.6, 8.5, 8.3, 8.2, 8.1, 8.0, 7.9, 7.8, 7.7, 7.6, 7.5, 7.4, 7.3, 7.2, 7.1, 7.0, 6.9, 6.8, 6.7, 6.6, 6.5, 6.4, 6.3, 6.2, 6.1, or 6.0. The end pH may range from 6.0 to 9.5.

In addition, autograft chips may be saturated with blood, bone marrow aspirate, bone-morphogenetic proteins, platelet-rich plasma, or osteogenic proteins to enhance bone regeneration. Some chips may require pretreatments longer than 24 hours. As used here in the context of pre-treatment time, the term “about” means +/−30 minutes. A person skilled in the art can determine other materials or solutions to be added to enhance bone regeneration.

In another embodiment of this aspect, silicate ions are released into and/or onto the bone defect. In yet another embodiment of this aspect, calcium ions are released into the bone. In a further embodiment, borate ions are released into the bone. In any of the above embodiments of this aspect of the invention, sufficient ions, which include but are not limited to silicate, calcium, and borate, are released from the bioactive glass ceramic into the bone defect to achieve a critical concentration of ions to stimulate the proliferation and differentiation of an osteoblast.

The bioactive glass ceramic fiber, mesh, or other shape can further comprise a carrier or an additional graft extender. The other shape can include glass wool, a ball of glass wool, a fabric, or an autograft chip wrapped in bioactive glass ceramic fiber. The carrier may be one or more of hydroxyapatite and tricalcium phosphate. The bioactive glass may be in a granular form and comprise other materials as carriers as well.

In one embodiment of this aspect, the bone defect is a fracture. The bone treatment composition comprising an autograft wrapped in a bioactive glass composition can be used to repair fractures. The autograft chip wrapped in bioactive glass may be used in combination with a fracture fixation device, with exemplary devices including pins, screws, plates, and nails. The autograft chip wrapped in bioactive glass is effective to stimulate bone remodeling, bone formation, osteoblast activity, and hydroxyapatite formation.

In another embodiment of this aspect, silicate ions are released into and/or onto the bone defect. In yet another embodiment of this aspect, calcium ions are released into the bone. In a further embodiment, borate ions are released into the bone. In any of the above embodiments of this aspect of the invention, sufficient ions, which include but are not limited to silicate, calcium, and borate, are released from the autograft chip wrapped in bioactive glass ceramic into the bone defect to achieve a critical concentration of ions to stimulate the proliferation and differentiation of an osteoblast.

Another aspect of the invention provides for a bone defect treatment wrap. The wrap is comprised of an autograft chip and a bioactive glass sheet. The bioactive glass sheet comprises a mesh of bioactive glass fibers. The bioactive glass sheet is wrapped around the autograft chip and the bone defect to prevent the autograft chip from migrating from the bone defect.

In some embodiments, an autograft chip is attached to a bone at or near a site of a bone defect. The autograft chip is secured to the bone by wrapping the autograft chip and the bone with a bioactive glass sheet comprising a mesh of bioactive glass fibers. The bioactive glass sheet is wrapped around the autograft chip and the bone defect to prevent the autograft chip from migrating from the bone defect.

In some embodiments, the bioactive glass fibers forming the bioactive glass sheet may be arranged in a variety of structures to form a wrap. In these structures, the bioactive glass fibers may be random-pressed cotton-like structures, wool-like structures, randomly-oriented fibers, woven, knitted, warped knitted, and/or braided. The bioactive glass fivers can also form a mesh-like structure. The bioactive glass sheet may be formed such that it has a substantially greater length than width.

In any of the woven, knitted, warped knitted, or braided patterns, the bioactive glass fibers are in both a longitudinal and transverse orientation. For example, the longitudinal fibers may be interwoven with transverse fibers. Some transverse fibers can be wrapped around the outside of the longitudinal fibers to secure the longitudinal and transverse fibers. In one example, the transverse fibers may be wrapped around the longitudinal fibers to form a knot or whipping. Alternatively, the longitudinal fibers may be stitched to the transverse fibers.

The openings within the sheet or mesh may have a low density. The structure and density of the bioactive glass fibers may be similar to the density of material in VICRYL (Ethicon, Inc., Somerville, N.J.). The sheet or mesh may have any one or more of about 5%, 10%, 15%, 20%, 25%, 30%, 35%, 40%, 45%, 50%, 55%, 60%, 65%, 70%, 75%, 80%, 85%, 90%, and 95% empty opening as a percentage of the area. The density of the mesh or sheet may be sufficiently high, i.e. the openings must have a low enough percentage area, such that the wrap is able to be sutured. The density may also be high enough such that the wrap serves as a barrier to hyperplasis and tissue adhesion.

The bioactive glass sheet or mesh may have binding regions, which may enable the mesh or sheet to be anchored to the bone. A bone anchoring device can affix or anchor the sheet or mesh by extending through the sheet or mesh. Exemplary bone anchoring devices include screws, sutures, stables, pins, buttons, and combinations thereof. The bone anchoring device can be attached to a drilled or hollowed out region of bone.

The bone defect may be a fracture or may result from an injury or bone disease, such as osteoporosis. The bioactive glass sheet may release one or more of silicate, calcium, and borate ions into the bone defect. Ions released into the bone defect can stimulate osteoblast activity. The bioactive glass sheet may further comprise a carrier, such as hydroxyapatite and tricalcium phosphate, or a graft extender.

Another aspect of the invention provides for a method for treating a bone defect using an autograft chip secured to the bone near the site of the bone defect by wrapping the bone and the autograft chip with a bioactive glass sheet.

Another aspect of the invention provides for treating a bone defect using an expandable empty bag comprising a mesh of bioactive glass fibers. The area of bone having a defect is exposed. At least one cavity is formed in the bone at or near the bone defect, in which a portion of the at least one cavity defines an opening. An expandable empty bag comprising a mesh of bioactive glass fibers is inserted into the opening.

The bag is packed with an autograft chip and optionally one or more of calcium, cement, a bone cement, and polymethyl methacrylate, until the bag expands to form a self-retaining rigid shape.

The bioactive glass ceramic provided by this aspect of the invention may be effective to produce hydroxyapatite in a hard tissue. An exchange of ions may occur between bioactive glass and the surrounding body fluid that results in the production of hydroxyapatite. The ions exchanged may be any one or more of silicate, calcium, and borate.

Another aspect of the invention provides a method for treating a bone defect. An area of bone having a defect is exposed. Such exposure may occur by drilling into the bone and then using a reamer in the bone. At least one cavity in the bone is formed at or near the bone defect such that a portion of the at least one cavity defines an opening. An expandable empty bag comprising a mesh of bioactive glass fibers is inserted into the opening. The bag is packed with an autograft chip and optionally one or more of calcium, cement, a bone cement, and polymethyl methacrylate, until the bag expands to form a self-retaining rigid shape.

The bag may be woven or form-molded to a density such that the autograft chip is retained while body fluids and solutions can enter and exit. In most instances, the pores of the bioactive glass mesh will have a diameter of about 0.01 mm to about 5.0 mm. For example, the pore may have a diameter of about 0.01 mm, 0.02 mm, 0.03 mm, 0.04 mm, 0.05 mm, 0.06 mm, 0.07 mm, 0.08 mm, 0.09 mm, 0.10 mm, 0.2 mm, 0.3 mm, 0.4 mm, 0.5 mm, 0.6 mm, 0.7 mm, 0.8 mm, 0.9 mm, 1.0 mm, 1.1 mm, 1.2 mm, 1.3 mm, 1.4 mm, 1.5 mm, 1.6 mm, 1.7 mm, 1.8 mm, 1.9 mm, 2.0 mm, 2.1 mm, 2.2 mm, 2.3 mm, 2.4 mm, 2.5 mm, 2.6 mm, 2.7 mm, 2.8 mm, 2.9 mm, 3.0 mm, 3.1 mm, 3.2 mm, 3.3 mm, 3.4 mm, 3.5 mm, 3.6 mm, 3.7 mm, 3.8 mm, 3.9 mm, 4.0 mm, 4.1 mm, 4.2 mm, 4.3 mm, 4.4 mm, 4.5 mm, 4.6 mm, 4.7 mm, 4.8 mm, 4.9 mm, or 5.0 mm. The bag is filled with one or more autograft bone chips. The autograft bone chips may be of various sizes.

Wrapping an autograft chip in a bag formed of bioactive glass mesh provides for several advantages. The autograft chip would not tend to flow out of a bone cavity, or an intervertebral cavity because the bioactive glass mesh walls would retain the autograft chip. The bag may be used in the posterolateral gutter to promote fusion and to secure the bone graft to the spine.

Another aspect of the invention provides for a method for treating a comminuted or complex bone fracture. A bone at the site of the comminuted or complex bone fracture is wrapped with a bioactive glass ceramic. The bioactive glass ceramic may be in the form of fibers, a fiber mesh, random fibers, oriented fibers, and/or a sheet.

The ceramic may be wrapped completely around the bone such that the ceramic is secured to the bone and/or maintains the bone shape so as to prevent further fracturing. One exemplary form of the bioactive glass ceramic is in the form of a mesh that can be wrapped around a large portion of bone surrounding the compound fracture so as to both provide pressure to the bone and to allow for the migration of ions from the mesh wrap into the bone. The bioactive glass ceramic may also be secured to the bone by one or more plates and/or one or more screws.

In some embodiments of any of the above aspects of the invention, the bioactive glass compositions may contain radioactive materials. Such bioactive glass compositions may be useful to treat tumors and bone defects arising out of cancer. The radiation emitted from the bioactive glass composition is effective to kill cancer cells within the tumors and bone defects.

### EXAMPLES

### Example 1

### Preparation of Bioactive Glass Ceramic

40 grams of quartz silica, 20 grams of phosphorous pentoxide, 3 grams of calcium carbonate, and 3 grams of sodium carbonate is mixed together. The mixture is then placed in a platinum crucible and melted at 1440° C. for 1.5 hours and poured into demineralized water to produce a granular glass frit. The bioactive glass frit is dried and ground in a mill to produce a powder. The powder is sieved through a 0.2 micron mesh sieve.

### Example 2

### Preparation of Bioactive Glass Fibers

The above ceramic is further treated by pulling molten ceramic into fibers. The fibers are then mixed together and pressed to form a mesh.

A bioactive glass ceramic prepared above is made into fibers by spinning. In spinning, heating treatment steps are performed, followed by mechanical pulling of the fibers. Techniques for spinning and pulling fibers are well known.

The fibers are tested by immersing them in Tris for three, five and seven days, respectively. Clear precipitation of calcium phosphate occurs after three days. The precipitation occurs as large flakes and starts to decay at seven days. The precipitations are evenly distributed at the surface of the fibers and this shows the high uniformity of the material.

### Example 3

### Weaving of Bioactive Glass Fibers to Form a Mesh

Bundles of bioactive glass fibers are prepared by manually weaving the fibers into a simple biaxial pattern. Specific procedures for weaving different patterns are numerous and widely available to those skilled in the art. The woven mesh is cleaned in isopropanol and dried in air. The micropore size and the distance between bundles are in the range of 150-200 μm and 400-800 μm.

### Example 4

### Testing of Bioactive Glass Ceramic In Vitro

The bioactive glass ceramic prepared above is tested by mixing 0.075 g of the powder with 50 mL of simulated body fluid. A layer of calcium carbonated apatite forms on the surface in six hours, as confirmed by X-ray powder diffraction and Fourier Transform Infrared Spectroscopy.

### Example 5

### Testing of Bioactive Glass Ceramic in Cell Culture

Bioactive glass ceramic, as prepared above, is incubated overnight in DMEM media at 37° C. and 5% CO₂ before culture. SAOS-2 cells, which are osteoblasts obtained from a cancer cell line, is cultured in DMEM medium containing 10% FBS, 1% L-glutamine, and 1% antiobiotic/antiomycotic and seeded at 10,000 cells/cm² in both a bioactive glass substrate and in a control substrate.

Alkaline phosphatase is then assayed in both the bioactive glass and control cultures according to the protocol described in Ball et al. Biomaterials, 2001, 22(4): 337-347. The SAOS-2 cells produce significantly more alkaline phosphatase in the presence of bioactive glass, as compared to the control.

### Example 6

### In Vivo Testing of Bone Graft Materials

An autograft bone chip from each of 18 canines is wrapped in a bioactive glass ceramic prepared above. Graft material that does not contain bioactive glass will serve as control. The test and control material are implanted into two bilateral drill defects that are surgically created in the cancellous bone of the proximal humerus of 18 canines. A test material is implanted into one of the drill defects in the first humerus, while a control article is implanted into the other drill defect in the opposite humerus. The drill defects will be approximately 10 mm in diameter and approximately 25 mm in depth.

Animals will be sacrificed and the implantation sites harvested and analyzed at various predetermined time points. Analysis includes mechanical testing to assess the bony ingrowth and remodeling of the defect site. The extent of bone remodeling and healing within the drill defects are characterized by the histopathological and histomorphometry evaluation. Defects treated with the bioactive bone graft ceramic show improved healing at each of the predetermined time points.

### Example 7

### Clinical Testing of Bone Graft Materials

An autograft bone chip from each of 25 human subjects is wrapped in a bioactive glass ceramic fiber as prepared according to Example 2. Graft material that does not contain bioactive glass will serve as control. The test and control material are implanted into two bilateral drill defects that are surgically created, one defect on the left side of the iliac crest and one defect on the right side of the iliac crest. The test material is planted into the left iliac crest and the control material was planted into the right iliac crest defect. At 12 weeks, all of the bone defects treated with test material had healed in each of the 25 subjects. At 15 weeks, 18 of the 25 bone defects treated with control material had healed in the 25 subjects.

### Example 8

### Implanting a Bag Containing an Autograft Chip into a Spinal Bone Defect

An expandable empty bag suitable for treating a bone defect in the spine is selected. A cavity is then formed in a bone at the site of the bone defect in the spine. The bag is then inserted into the cavity.

The bag is filled with enough ground autograft chip material to fill the bag such that the bag assumes and retains a rigid shape. The bag is not completely filled if the shape of the bag with material can be easily deformed. The bag is sealed and an assessment is performed to determine if any portion of the autograft chip filler leaks from the bag.

Throughout this specification various indications have been given as to preferred and alternative embodiments of the invention. However, the foregoing detailed description is to be regarded as illustrative rather than limiting and the invention is not limited to any one of the provided embodiments. It should be understood that it is the appended claims, including all equivalents, that are intended to define the spirit and scope of this invention.

1. A bone defect treatment composition comprising:
a bone graft comprising a natural or synthetic graft material, or a combination thereof; and
a bioactive glass ceramic having a form selected from the group consisting of fibers, a fiber mesh, random fibers, oriented fibers, and a sheet;
wherein the bioactive glass ceramic is wrapped around the bone graft and prevents the bone graft from migrating from the bone defect.

2. The composition of claim 1, wherein the bioactive glass ceramic comprises SiO₂.

3. The composition of claim 1, wherein the bioactive glass ceramic comprises P₂O₅, PO₃. or PO₄.

4. The composition of claim 1, wherein the bioactive glass ceramic comprises B₂O₃.

5. The composition of claim 1, wherein the bioactive glass ceramic comprises 40-60% SiO₂, 10-20% CaO, 0-4% P₂O₅, and 19-30% Na₂O.

6. The composition of claim 1, wherein the bioactive glass ceramic further comprises a carrier selected from the group consisting of hydroxyapatite and tricalcium phosphate.

7. The composition of claim 1, wherein the bioactive glass ceramic is in the form of a mesh.

8. The composition of claim 1, wherein the bioactive glass ceramic is in the form of a sheet.

9. The composition of claim 1, wherein the bioactive glass ceramic is in the form of fibers having a diameter of between about 10 nm and about 10,000 nm.

10. The composition of claim 1, wherein the bone graft and the bioactive glass ceramic are pretreated in a solution comprising one or more of blood, bone marrow aspirate, bone-morphogenetic proteins, platelet-rich plasma, and osteogenic proteins.

11. The composition of claim 1, wherein the bioactive glass ceramic does not include any substantial amount of polymer.

12. A method for wrapping a bone graft applied to a bone defect comprising wrapping the bone graft with a bioactive glass ceramic having a form selected from the group consisting of fibers, a fiber mesh, random fibers, oriented fibers, and a sheet, wherein the bioactive glass ceramic prevents the bone graft from migrating from the bone defect.

13-27. (canceled)

28. A bone defect treatment wrap comprising:
a bone graft; and
a bioactive glass sheet comprising a mesh of bioactive glass fibers;
wherein the bioactive glass sheet is wrapped around the bone graft and the bone defect to prevent the bone graft from migrating from the bone defect.

29. The wrap of claim 28, wherein the bioactive glass fibers are present in both a longitudinal and transverse pattern within the mesh.

30. The wrap of claim 28, wherein the bioactive glass fibers form a structure selected from the group consisting of woven, knitted, lace-like, non-woven, and braided.

31. The wrap of claim 28, wherein the bioactive glass fibers comprise SiO₂.

32. The wrap of claim 28, wherein the bioactive glass fibers comprise P₂O₅, PO₃. or PO₄.

33. The wrap of claim 28, wherein the bioactive glass fibers comprise B₂O₃.

34. The wrap of claim 28, wherein the bioactive glass fibers comprise 40-60% SiO₂, 10-20% CaO, 0-4% P₂O₅, and 19-30% Na₂O.

35. The wrap of claim 28, wherein the bioactive glass fibers further comprise a carrier selected from the group consisting of hydroxyapatite and tricalcium phosphate.

36. The wrap of claim 28, wherein the bioactive glass fibers have a diameter of between about 10 nm and about 10,000 nm.

37. The wrap of claim 28, wherein the bone graft and the bioactive glass fibers are pretreated in a solution comprising one or more of blood, bone marrow aspirate, bone-morphogenetic proteins, platelet-rich plasma, and osteogenic proteins.

38. The wrap of claim 28, wherein the bioactive glass fibers do not include any substantial amount of polymer.

39-65. (canceled)
